(19) United States
(12) Patent Application Publication
(10) Pub. No.: US 2017/0354575 A1
(43) Pub. Date: Dec. 14, 2017

(54) SYSTEM AND DEVICE FOR SUPPORTING A FOOD DISPENSER AND USES THEREOF

(71) Applicant: Shannon Kelly Pryor, Toms River, NJ (US)

(72) Inventor: Shannon Kelly Pryor, Toms River, NJ (US)

(21) Appl. No.: 15/620,777

(22) Filed: Jun. 12, 2017

Related U.S. Application Data
(60) Provisional application No. 62/349,581, filed on Jun. 13, 2016.

(51) Int. Cl. A61J 9/06; F16M 11/04; A61M 5/14; A61G 7/05; A61G 5/10

(52) U.S. Cl. CPC A61J 9/0638; A61G 7/0503; A61G 5/10; A61M 5/1415; A61J 9/0661; F16M 11/041; F16M 11/046; A61J 2200/70; A61G 2205/00

(57) ABSTRACT

The present invention is related to a device for supporting a food dispenser. The device includes a strap. The strap includes a first end and a second end opposite to the first end. The strap further includes a plurality of openings disposed between the first end and the second end. The strap also includes a plurality of markings corresponding to the plurality of openings. Each of the plurality of markings is indicative of a distance of the corresponding opening from the first end or the second end. The device also includes a retaining member detachably secured within one of the plurality of openings. The retaining member is configured to secure the food dispenser to the strap.

### CROSS-REFERENCE TO RELATED APPLICATIONS

This application claims the benefit of U.S. Provisional Application Ser. No. 62/349,581 filed on Jun. 13, 2016, the entire content of which is hereby incorporated by reference in its entirety.

### FIELD OF INVENTION

Embodiments of the present invention relate to systems and devices for supporting a food dispenser.

### BACKGROUND

Feeding tubes are vital for the survival and recovery of patients who are unable to eat orally or receive proper nutrition by swallowing. In such conditions, food, interchangeably referred to as nutrition, is directly fed to the patients by means of a food dispenser attached to a feeding tube going into their stomach or small intestines, usually regulated and administered by gravity.

There are several support systems in the art for holding gravity food dispensers. A gravity food dispenser can be in the form of a syringe, bag or like feed container attached to a feeding tube, which is then secured and positioned at a certain height designating the rate for feeding a patient. If a feed is administered at a high rate, it can result in vomiting, thereby causing pain and discomfort to the patient. Generally, while administering the feed, syringe, bag or like feed container is hung by an adhesive tape to a stationary object or piece of equipment. When the adhesive tape is removed, often a sticky residue is left behind on the object and/or the equipment. The sticky residue may aid in the collection and growth of bacteria and germs. The germs or bacteria can further lead to transmission of various diseases and illness to the patient or those in the vicinity of the patient. In some instances, the feeding tubes are held manually by medical personnel or any other person for the duration of the feed for upwards of 45-60 minutes, which further causes discomfort to the patient due to inconsistent height and fatigue to those individuals holding the feeding device.

Accordingly, there is a need for support systems that provide sanitary conditions during nutritional administration.

There is a further need for support systems that can hold or secure a food dispenser while maintaining a consistent rate of feeding to eliminate feeds being administered too quickly, and thereby relieving patients of discomfort and vomiting associated with rapid feeds.

### SUMMARY

Embodiments in accordance with the present invention provide a support system for supporting a food dispenser, the support system including an adjustable gravity strap that can be secured to a stationary item or a patient allowing for hands free administration.

Embodiments in accordance with the present invention provide a support system for supporting a food dispenser, the support system including an adjustable gravity strap that aids in retaining the food dispenser at a desired height, which eliminates food and nutrition, including but not limited to fluids, hydration and medication, being administered too quickly and relieves a patient of discomfort and vomiting associated with rapid feedings.

Embodiments in accordance with the present invention provide a support system for supporting a food dispenser, the support system including an adjustable gravity strap that reduces the exposure to bacteria and germs during feedings as the food dispenser is no longer taped to stationary items, such as cribs or chairs. The support system may therefore eliminate residues left behind by removal of the tape that can otherwise act as breeding grounds for bacteria.

Embodiments in accordance with the present invention provide a support system for supporting a food dispenser, the support system including an adjustable gravity strap that is substantially latex free.

Embodiments in accordance with the present invention provide a support system for supporting a food dispenser, the support system including an adjustable gravity strap that is washable and re-usable.

Embodiments in accordance with the present invention provide a support system for supporting a food dispenser, the support system including an adjustable gravity strap that is disposable.

Embodiments in accordance with the present invention provide a device for supporting a food dispenser. The device includes a strap. The strap includes a first end, and a second end opposite to the first end. The strap also includes a plurality of openings disposed between the first end and the second end. The strap further includes a plurality of markings corresponding to the plurality of openings, each of the plurality of markings being indicative of a distance of the corresponding opening from the first end or the second end. The device further includes a retaining member detachably secured within one of the plurality of openings. The retaining member is configured to secure the food dispenser to the strap.

Embodiments in accordance with the present invention are directed to a system for supporting a food dispenser, the system including a stand and a strap. The strap includes a first portion detachably coupled to the stand. The first portion includes a first end of the strap. The strap also includes a second portion spaced apart from the first portion. The second portion includes a second end of the strap. The strap further includes a third portion disposed between the first portion and the second portion. The third portion includes a plurality of openings spaced apart from each other. The third portion also includes a plurality of markings corresponding to the plurality of openings. Each of the plurality of markings is indicative of a distance of the corresponding opening from the first end of the strap or the second end of the strap. The system also includes a retaining member detachably secured within one of the plurality of openings, wherein the retaining member is configured to secure the food dispenser to the strap.

Embodiments in accordance with the present invention are further directed to a device for supporting a food dispenser. The device includes a strap including a first elongate member. The strap also includes a second elongate member attached to the first elongate member at a plurality of attachment regions spaced apart from each other. The first elongate member and the second elongate member define a plurality of openings therebetween. Further, each of the plurality of openings is located between two adjacent attachment regions. The strap further includes a plurality of markings corresponding to the plurality of openings. Each of the plurality of the markings is indicative of a distance of the corresponding opening from a first end of the strap or a second end of the strap. The device also includes a retaining member detachably secured within one of the plurality of openings. The retaining member is configured to secure the food dispenser to the strap.

These and other advantages will be apparent from the present application of the embodiments described herein.

The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

### BRIEF DESCRIPTION OF THE DRAWINGS

The accompanying drawings, which are incorporated in and constitute a part of this specification, illustrate several embodiments of the disclosure and, together with the description, explain the principles of the disclosure.

FIG. 1 illustrates a front view of a system for supporting a food dispenser, according to an embodiment of the present invention;

FIG. 2 illustrates a side view of a strap, according to an embodiment of the present invention;

FIG. 3 illustrates a front view of the system for supporting a food dispenser, according to an embodiment of the present invention;

FIG. 4 illustrates a side view of a food dispenser secured to a strap, according to an embodiment of the present invention;

FIG. 5 illustrates a method of supporting a food dispenser, according to an embodiment of the present invention;

While embodiments of the present disclosure are described herein by way of example using several illustrative drawings, those skilled in the art will recognize the present disclosure is not limited to the embodiments or drawings described. It should be understood the drawings and the detailed description thereto are not intended to limit the present disclosure to the form disclosed, but to the contrary, the present disclosure is to cover all modification, equivalents and alternatives falling within the spirit and scope of embodiments of the present disclosure as defined by the appended claims.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

### DETAILED DESCRIPTION

Embodiments of the present invention will be illustrated below in conjunction with exemplary configurations of a system and a device for supporting a food dispenser.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The terms “first,” “second,” and the like, herein do not denote any order, quantity, or importance, but rather are used to distinguish one element from another.

The term “IV pole” refers to poles which have hooks to support intravenous (IV) equipment.

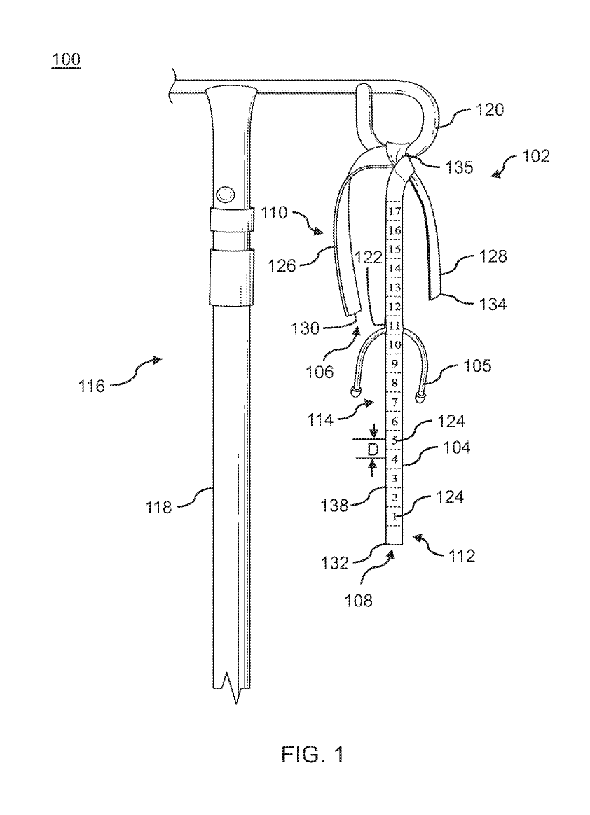

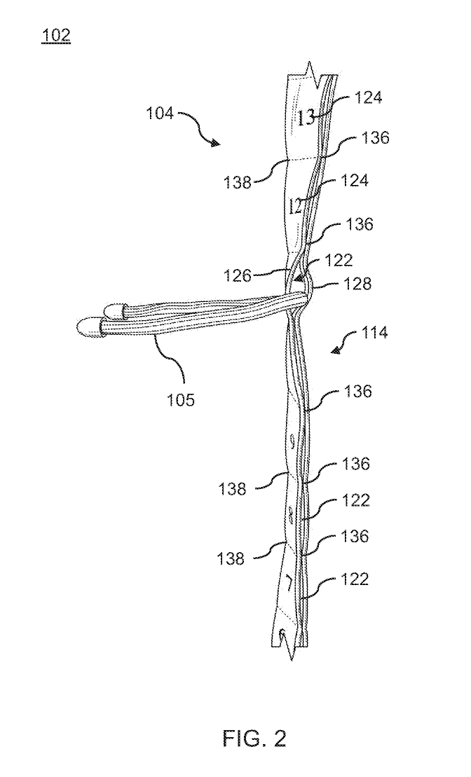

FIG. 1 is a front view of a support system 100 (hereinafter referred to as “the system 100”) for supporting a food dispenser (not shown in FIG. 1), according to an embodiment of the present invention. The system 100 includes a device 102 including an adjustable gravity strap 104 (hereinafter referred to as “the strap 104”) and a detachable retaining member 105 (hereinafter referred to as “the retaining member 105”). FIG. 2 illustrates a side view of the strap 104.

The strap 104 is configured to support the food dispenser at a predetermined height. In some embodiments, the food dispenser can include, but not limited to, a syringe, a feeding tube, a feeding bag, a feeding pouch, and the like. The predetermined height can be a height of the food dispenser with reference to a ground surface, a patient, or an article on which the patient is resting, for example, the patient's bed, chair, and the like. In another embodiment, the predetermined height may be a height of the food dispenser relative to the patient's stomach. The predetermined height may determine the rate of feeding, i.e., the rate at which the food or nutrition is provided to the patient.

Referring to FIGS. 1 and 2, the strap 104 includes a first end 106 and a second end 108 opposite to the first end 106. The first and second ends 106, 108 are longitudinal ends of the strap 104. Further, the strap 104 includes a first portion 110, a second portion 112 spaced apart from the first portion 110, and a third portion 114 disposed between the first portion 110 and the second portion 112. The first portion 110 includes the first end 106 of the strap 104, while the second portion 112 includes the second end 108 of the strap 104. In some embodiments, the first portion 110 may be detachably coupled to a stand 116. In an embodiment, the stand 116 is an IV pole. In some embodiments, the stand 116 may include a pole 118, a base (not shown) with one or more legs and a hanger 120. The pole 118 may have a telescoping structure which permits height adjustment of the stand 116. The stand 116 may also be secured to a structure, such as a bed, a stretcher, a wheelchair, or a table. In some embodiments, the first portion 110 may include two strips which are tied together with the hanger 120 of the stand 116. In some other embodiments, the first portion 110 may be detachably coupled to the stand 116 using any suitable fastening means such as, but not limited to, a Velcro strap, a twisted tie, and so forth. In alternative embodiments, the first portion 110 of the strap 104 may be detachably secured to a body part of the patient. In an example, the first portion 110 may be secured to a finger of the patient.

In an exemplary embodiment, the third portion 114 includes multiple openings 122 spaced apart from each other along a length of the third portion 114. In some embodiments, the openings 122 may be equally spaced from each other by a distance ‘D’. In some other embodiments, the openings 122 may be unequally spaced along the third portion 114. The third portion 114 further includes multiple markings 124 corresponding to the multiple openings 122. The markings 124 may be printed on the strap 104 by any suitable method such as, but not limited to, jet printing. In some other embodiments, the markings 124 may embossed on the strap 104. In an exemplary embodiment, the markings 124 are numbers. In some other embodiments, the markings 124 may include characters, symbols, or a combination thereof. Further, each of the markings 124 is indicative of a distance or height of the corresponding opening 122 relative to a reference point. Specifically, when the strap 104 is suspended from an object, each of the markings 124 may indicate a height of the corresponding opening 122 from the reference point. In some embodiments, the reference point may be the first end 106 of the strap 104. In other embodiments, the reference point may be the second end 108 of the strap 104. In yet other embodiments, the reference point may be a ground surface, a part of the patient's body, or an article on which the patient is resting, for example, the patient's bed, chair, and the like. In some embodiments, the distance indicated by the each of the markings 124 may be defined in any suitable unit such as, but not limited to, inches, centimeters, millimeters and so forth. In another embodiment, each of the markings 124 is a number that may indicate a distance in inches from the second end 108 of the strap 104. Therefore, when the strap 104 is suspended from an object, each of the markings 124 may indicate a height of the corresponding opening 122 from the second end 108 of the strap 104.

In some embodiments, the strap 104 includes a first elongate member 126 and a second elongate member 128. The first elongate member 126 includes a first end 130 and a second end 132 opposite to the first end 130. In an embodiment, the first end 130 of the first elongate member 126 may coincide with the first end 106 of the strap 104. The second end 132 of the first elongate member 126 may similarly coincide with the second end 108 of the strap 104. The second elongate member 128 includes a first end 134. In some embodiments, the first end 134 of the second elongate member 128 may coincide with the first end 106 of the strap 104. Further, a second end (not shown) of the second elongate member 128 may coincide with the second end 108 of the strap 104. In an exemplary embodiment, the first elongate member 126 may be attached to the second elongate member 128 in a manner, such that the first end 130 of the first elongate member 126 and the first end 134 of the second elongate member 128 are separated from each other. In a further embodiment, the first elongate member 126 and the second elongate member 128 may be detached or separated from each other in the first portion 110 of the strap 104. Therefore, the separated portions of the first and the second elongate members 126, 128 may allow the strap 104 to be tied and secured to the stand 116. In the illustrated embodiment, the first and second elongate members 126, 128 are tied to the hanger 120 of the stand 116 via a knot 135. In some other embodiments, the separated first ends 130, 134 may be tied to a body part of the patient, or an article on which the patient is resting, for example, the patient's bed, chair, and the like. In alternative embodiments, the first end 130 of the first elongate member 126 may be attached to the first end 134 of the second elongate member 128 by any suitable attachment method, such as, but not limited to, Velcro, adhesives, sewing, snap fastener, and so forth.

In an embodiment, the second elongate member 128 is attached to the first elongate member 126 at multiple attachment regions 136 spaced apart from each other. The multiple attachment regions 136 may be located in the third portion 114 of the strap 104. The second elongate member 128 may be coupled to the first elongate member 126 at the attachment regions 136 by any suitable attachment methods, such as, but not limited to, sewing, adhesive, and so forth. Further, the first elongate member 126 and the second elongate member 128 define the multiple openings 122 therebetween. Each of the openings 122 is located between two adjacent attachment regions 136. Further, the attachment regions 136 may coincide with multiple marking lines 138. Each of the markings 124, that indicates the distance of the corresponding opening 122, is located between two adjacent marking lines 138. The markings 124 and the marking lines 138 may be printed on the first elongate member 126 and/or the second elongate member 128. In some other embodiments, the markings 124 and the marking lines 138 may be embossed on the first elongate member 126 and/or the second elongate member 128.

In an exemplary embodiment, each of the markings 124 indicates a distance of the corresponding openings 122 from the second end 132 of the first elongate member 126. In some other embodiments, each of the markings 124 indicates a distance of the corresponding opening 122 from the first end 130 of the first elongate member 126. Alternatively, each of the markings 124 indicates a distance of the corresponding opening 122 from the first end 134 or the second end of the second elongate member 128.

In an embodiment, the second end 132 of the first elongate member 126 and the second end of the second elongate member 128 are attached to each other. In a further embodiment, the first elongate member 126 and the second elongate member 128 may be attached to each other in the second portion 112 of the strap 104. In alternative embodiments, the second end 132 of the first elongate member 126 and the second end of the second elongate member 128 are separated to each other. Specifically, the first elongate member 126 and the second elongate member 128 may be separated from each other in the second portion 112 of the strap 104, thereby allowing the strap 104 to be secured to a fixed structure or the patient at the second end 108. In some embodiments, the first elongate member 126 and the second elongate member 128 may have substantially equal lengths. Alternatively, the lengths of the first elongate member 126 and the second elongate member 128 may be different.

In some other embodiments, the strap 104 may include a single elongate member (not shown) which includes a plurality of punched holes or openings. Each of the punched holes may be located at a predetermined distance. The predetermined distance may correspond to a height of the hole with reference to a ground surface, a patient, or an article on which the patient is resting, for example, the patient's bed, chair, and the like. The single elongate member may also include a Velcro strap at the first end 106 and/or the second end 108 which allows the strap 104 to be tied or secured to a fixed structure.

In some embodiments, the strap 104 may be manufactured from a latex free material to prevent allergic reactions to the patient. Further, the material of the strap 104 may include foam such as, but not limited to, polyethylene foam, polyurethane foam, and the like. In some embodiments, the strap 104 is washable and re-usable. In other embodiments, the strap 104 is disposable.

In some embodiments, the strap 104 may be a double tie-strap.

The device 102 further includes the retaining member 105. The retaining member 105 is configured to be detectably secured within one of the openings 122. The retaining member 105 may secure the food dispenser to the strap 104. In an exemplary embodiment, the retaining member 105 is a twist tie. The retaining member 105 may be made of metal or plastic. However, the retaining member 105 may include any type of fastener that can detachably secure the food dispenser to the strap 104. Examples of such fasteners may include, but not restricted to, strings, clips, Velcro straps, and the like.

Further, the retaining member 105 may be deformed to secure food dispensers of different dimensions (e.g., diameters) to the strap 104.

In some embodiments, the device 102 may include a plurality of retaining members secured within multiple openings 122 of the strap 104.

Further, as illustrated in FIGS. 1 and 2, the retaining member 105 is disposed in the opening 122 at a central section of the third portion 114. Specifically, the retaining member 105 is disposed in the opening 122 with the marking “11” indicating that the retaining member 105 may be located approximately at a height of 11 inches from the second end 108 of the strap 104. However, the retaining member 105 may be disposed in any of the openings 122 depending upon a desired feeding rate of the food dispenser secured to the retaining member 105. For example, an increase in height (e.g., 13 inches) from the second end 108 may increase the feeding rate of the food dispenser. A decrease in height (e.g., 10 inches) from the second end 108 may decrease the feeding rate. Therefore, the strap 104 may allow adjustment of the feeding rate so that an optimal feeding rate can be chosen for the patient. Further, the feeding rate may be consistent during administration of the feed since the feed dispenser is retained at a fixed height. An optimal and consistent feeding rate may prevent vomiting, pain and discomfort to the patient.

The strap 104 can be secured to a stationary item (e.g., a stand, a crib, a chair, etc.) or a person. Further, the food dispenser is secured to the strap 104 without any tape that can leave a residue. Therefore, the system 100 may reduce exposure to bacteria and germs during feeds, as the residues left behind by the tape are eliminated. Further, the strap 104 improves comfort and efficiency of medical personnel, such as nurses, as the strap 104 may eliminate manual retention of the food dispenser during feedings. The strap 104 may also benefit patients by not requiring the patients to hold or support the food dispenser. With the use of the system 100, medical personnel can spend more time focusing on the patients' needs as the system 100 enables hands free administration of the feed. In addition, the pre-printed measurements on the strap 104 may aid in ensuring accuracy of feeding between nursing shifts.

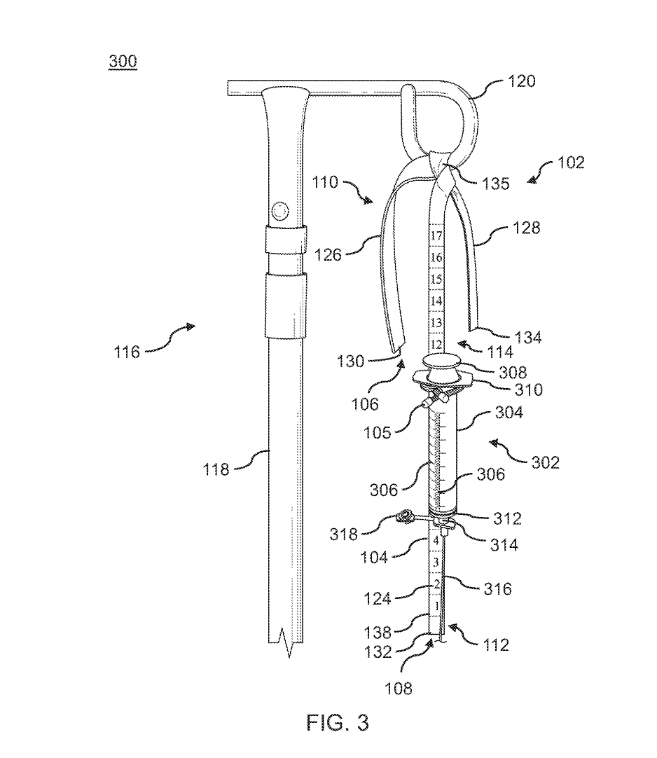

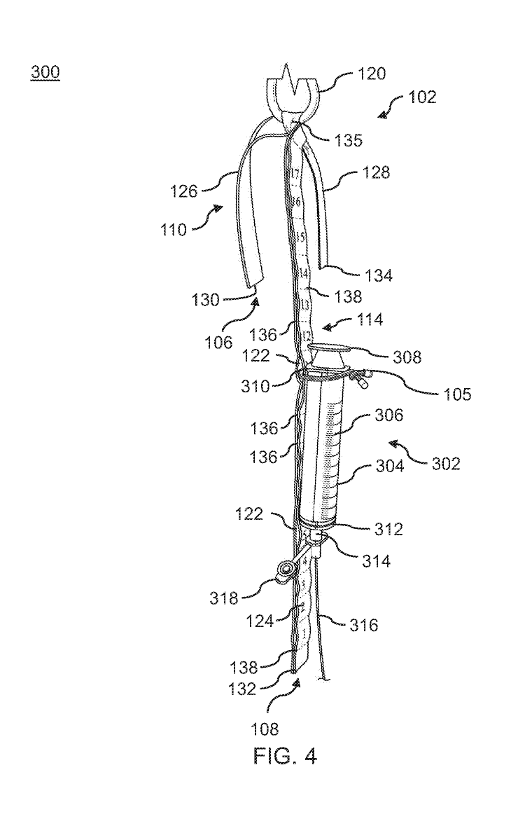

FIG. 3 is a front view of a support system 300 (hereinafter referred to as “the system 300”) with a food dispenser 302 secured to the strap 104. FIG. 4 is a lateral view of the support system 300. Reference numerals corresponding to similar components in FIGS. 1 and 2 have been used in FIGS. 3 and 4. Therefore, various components of the system 100 have not been described with reference to FIGS. 3 and 4.

The system 300 includes the device 102 including the strap 104 and the retaining member 105. The strap 104 supports the food dispenser 302 at a predetermined height. In some embodiments, the food dispenser 302 can include, but not limited to, a syringe, a feeding tube, a feeding bag, a feeding pouch, and the like. The predetermined height can be a height of the device 102 with reference to a ground surface, a patient, or an article on which the patient is resting, for example, the patient's bed, chair, and the like. In another embodiment, the predetermined height may be a height of the food dispenser relative to the patient's stomach.

In an exemplary embodiment, the food dispenser 302 is a gravity food dispenser syringe. The food dispenser 302 includes a body 304 for storing a feed that is supplied to the patient. In some embodiments, the feed may include various type of intravenous fluids. In some other embodiments, the feed may be any nutritious material which is administered into a stomach or small intestines of the patient. As illustrated in FIG. 3, the body 304 is a hollow tubular structure. The body 304 includes a plurality of indicia 306 indicative of a volume of feed contained in the body 304. The food dispenser 302 further includes a plunger 308 at least partially received within the body 304. In some embodiments, the plunger 308 may be removed to fill the body 304 with the feed. In some other embodiments, the plunger 308 may be pulled towards a top end 310 of the body 304 to refill the food dispenser 302.

The outlet 314 of the food dispenser 302 may dispense the feed from the body 304. In some embodiments, the outlet 314 may be connected to a feeding tube 316. The feeding tube 316 may transport the feed from the food dispenser 302 to the patient. In some embodiments, the feeding tube 316 may be coupled to a needle or a catheter to transmit the feed into the patient's body.

In some embodiments, the food dispenser 302 is secured to the strap 104 via the retaining member 105 at a predetermined height. The predetermined height is determined based on a feeding rate suitable for the patient. In an exemplary embodiment, the retaining member 105 is a twisted tie which is twisted about the body 304 of the food dispenser. As illustrated in FIG. 3, the food dispenser 302 is secured to the strap 104 at a central section of the third portion 114. Specifically, the food dispenser 302 may be secured at the marking “11” (shown in FIG. 1) indicating that the food dispenser 302 is located approximately at a height of 11 inches from the second end 108 of the strap 104. However, the retaining member 105 may be disposed at any of the openings 122 depending upon a desired feeding rate of the food dispenser 302.

The retaining member 105 may secure the food dispenser 302 to any of the openings 122 located at suitable height such that a suitable rate of feeding is maintained, thereby relieving patients of discomfort and vomiting associated with rapid feeds.

In some embodiments of the present invention, multiple such food dispensers may be secured to multiple detachable retaining members disposed within the openings defined by the strap 104.

The food dispenser 302 can be easily detached from the detachable retaining member 105 for cleaning purposes to keep the food dispenser 302 disinfected.

The food dispenser 302 is secured by the strap 104 so as to prevent spilling or dropping of the food dispenser during feeding.

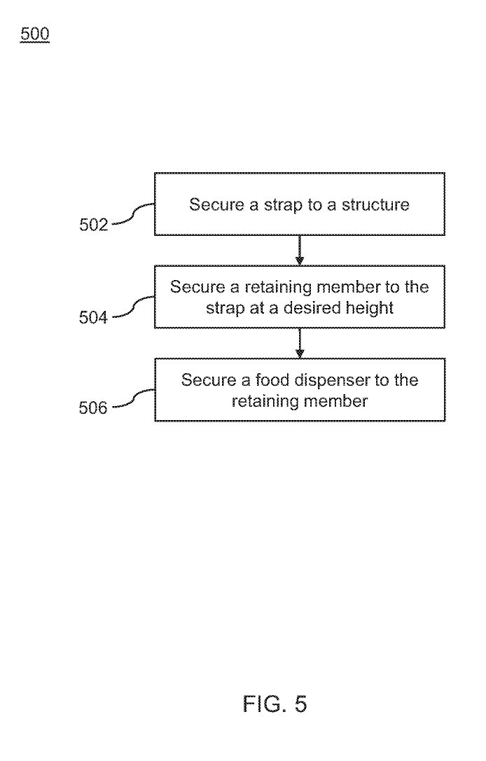

FIG. 5 shows flowchart illustrating a method 500 of supporting the food dispenser 302. At step 502, the strap 104 is secured to a structure. In an exemplary embodiment, the structure is the stand 116. Alternatively, the structure may be a body part of the patient, or an article on which the patient is resting, for example, the patient's bed, chair, and the like. In an exemplary embodiment, the separated ends of the first elongate member 126 and the second elongate member 128 of the strap 104 are tied to the hanger 120 of the stand 116 via the knot 135. Alternatively, the strap 104 may be coupled to the structure via other attachment means, such as, but not limited to, Velcro strap, adhesive, and so forth.

Next at step 504, the retaining member 105 is secured to the strap 104 via one of the openings 122. Specifically, the retaining member 105 is inserted within one of the openings 122 at a desired height. In an exemplary embodiment, the retaining member 105 is a twisted tie. However, the retaining member 105 may include any type of fastener that can detachably secure the food dispenser to the strap 104. Examples of such fasteners may include, but not restricted to, strings, clips, Velcro straps, and the like.

At step 506, the food dispenser 302 is secured to the strap 104 via the retaining member 105. The retaining member 105 may be wrapped around the food dispenser 302. Ends of the retaining member 105 may be then twisted to secure the food dispenser 302 to the strap 104. After administration of the feed, the food dispenser 302 may be detached from the retaining member 105.

Generally, the support system of the present invention provides an accurate, sanitary and simple solution for gravity feeding. However, the advantages of the support system are not limited to the ones described in the aforementioned embodiments.

The present invention, in various embodiments, configurations, and aspects, includes components, methods, processes, systems and/or apparatus substantially as depicted and described herein, including various embodiments, sub-combinations, and subsets thereof. Those of skill in the art will understand how to make and use the present invention after understanding the present disclosure. The present invention, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease and/or reducing cost of implementation.

The foregoing discussion of the present invention has been presented for purposes of illustration and description. It is not intended to limit the present invention to the form or forms disclosed herein. In the foregoing Detailed Description, for example, various features of the present invention are grouped together in one or more embodiments, configurations, or aspects for the purpose of streamlining the disclosure. The features of the embodiments, configurations, or aspects may be combined in alternate embodiments, configurations, or aspects other than those discussed above. This method of disclosure is not to be interpreted as reflecting an intention the present invention requires more features than are expressly recited in each claim.

Moreover, though the description of the present invention has included description of one or more embodiments, configurations, or aspects and certain variations and modifications, other variations, combinations, and modifications are within the scope of the present invention, e.g., as may be within the skill and knowledge of those in the art, after understanding the present disclosure. It is intended to obtain rights which include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A device for supporting a food dispenser, the device comprising:
a strap comprising:
a first end;
a second end opposite to the first end;
a plurality of openings disposed between the first end and the second end; and
a plurality of markings corresponding to the plurality of openings, each of the plurality of markings being indicative of a distance of the corresponding opening from the first end or the second end; and
a retaining member detachably secured within one of the plurality of openings, wherein the retaining member is configured to secure the food dispenser to the strap.

2. The device of claim 1, wherein the strap is detachably coupled to a stand.

3. The device of claim 1, wherein the strap comprises a material that is latex free.

4. The device of claim 3, wherein the material comprises foam.

5. The device of claim 1, wherein the strap further comprises:
a first elongate member; and
a second elongate member attached to the first elongate member at a plurality of attachment regions spaced apart from each other, wherein the first elongate member and the second elongate member define the plurality of openings therebetween.

6. The device of claim 5, wherein the first elongate member and the second elongate member are detached from each other at the first end of the strap.

7. The device of claim 1, wherein the retaining member comprises a twist tie.

8. The device of claim 1, wherein each of the plurality of markings comprise numbers.

9. A system for supporting a food dispenser, the system comprising:
a stand;
a strap comprising:
a first portion detachably coupled to the stand, the first portion comprising a first end of the strap;
a second portion spaced apart from the first portion, the second portion comprising a second end of the strap; and
a third portion disposed between the first portion and the second portion, the third portion comprising:
a plurality of openings spaced apart from each other; and
a plurality of markings corresponding to the plurality of openings, wherein each of the plurality of markings is indicative of a distance of the corresponding opening from the first end of the strap or the second end of the strap; and
a retaining member detachably secured within one of the plurality of openings, wherein the retaining member is configured to secure the food dispenser to the strap.

10. The system of claim 9, wherein the strap comprises a material that is latex free.

11. The system of claim 10, wherein the material comprises foam.

12. The system of claim 9, wherein the strap further comprises:
a first elongate member; and
a second elongate member attached to the first elongate member at a plurality of attachment regions spaced apart from each other, wherein the first elongate member and the second elongate member define the plurality of openings therebetween.

13. The system of claim 12, wherein the first elongate member and the second elongate member are detached from each other in the first portion of the strap.

14. The system of claim 9, wherein the retaining member comprises a twist tie.

15. A device for supporting a food dispenser, the device comprising:
a strap comprising:
a first elongate member;
a second elongate member attached to the first elongate member at a plurality of attachment regions spaced apart from each other, wherein the first elongate member and the second elongate member define a plurality of openings therebetween, each of the plurality of openings located between two adjacent attachment regions; and
a plurality of markings corresponding to the plurality of openings, each of the plurality of the markings being indicative of a distance of the corresponding opening from a first end of the strap or a second end of the strap; and
a retaining member detachably secured within one of the plurality of openings, wherein the retaining member is configured to secure the food dispenser to the strap.

16. The device of claim 15, wherein the strap comprises a material that is latex free.

17. The device of claim 16, wherein the material comprises foam.

18. The device of claim 15, wherein the retaining member comprises a twist tie.

19. The device of claim 15, wherein the strap is detachably coupled to a stand.

20. The device of claim 15, wherein the first elongate member and the second elongate member are detached from each other at the first end of the strap.
